(19) United States
(12) Patent Application Publication
(10) Pub. No.: US 2025/0162974 A1
(43) Pub. Date: May 22, 2025

(54) CARBONATE-CONTAINING UNSATURATED COMPOUND, PREPARATION METHOD THEREFOR, CURED PRODUCT PREPARED THEREFROM, AND METHOD FOR DEGRADING CURED PRODUCT

(71) Applicant: Swancor Innovation & Incubation Co., Ltd., Nantou County (TW)

(72) Inventors: Meng-Wei WANG, Nantou County (TW); Chun-An CHEN, Nantou County (TW); Meng-Ting CHANG, Nantou County (TW); Chia-Hao CHANG, Nantou County (TW); Ming-Yao CHENG, Nantou County (TW)

(21) Appl. No.: 18/844,392

(22) PCT Filed: Mar. 7, 2022
(86) PCT No.: PCT/CN2022/079570

§ 371 Date: Sep. 6, 2024

(51) Int. Cl. C07C 68/06; C08F 8/50; C08F 246/00

(52) U.S. Cl. CPC C07C 68/06; C08F 8/50; C08F 246/00

(57) ABSTRACT

The original abstract is amended to “A carbonate-containing unsaturated compound and a preparation method for a carbonate-containing unsaturated compound, a cured product and a method for degrading a cured product are provided. The carbonate-containing unsaturated compound includes a structure represented by formula (I) or formula (II). Formula (I) and formula (II) are defined as in the specification.”

### BACKGROUND

### Technical Field

The present disclosure relates to an unsaturated compound, a preparation method therefor, a cured product prepared therefrom and a method for degrading a cured product. More particularly, the present disclosure relates to a carbonate-containing unsaturated compound, a preparation method therefor, a cured product prepared therefrom and a method for degrading a cured product.

### Description of Related Art

The thermosetting materials have the characteristics of good processability before curing, and have the excellent thermal stability, the mechanical strength and the chemical resistance after cross-linking and curing. Therefore, the thermosetting materials are widely used in the various fields, and often used in the fiber composite materials with the high strength and lightweight requirements. However, due to the characteristics of non-melt-processable and good chemical resistance of the thermosetting materials, also causing that it is difficult to recycle and reuse them after waste. Moreover, the combustion of fiber composite materials is easy to shorten the life of the incineration equipment and cause the problem of large amount of waste. Therefore, how to deal with the waste thermosetting materials is an improvement goal in today's environmental issues.

At present, the vinyl ester resin and the unsaturated polyester resin are commonly used thermosetting materials in the industry, which are widely used in the flied, such as coating, transportation and construction, etc. Due to the booming development of automobiles, ships, or other mass transportation industries, the application market thereof continues to expand. For the applications and developers, the disposal of resin waste has become a major issue that has to be faced, so that the industry is eager to know the technologies that can decompose or recycle the resin waste.

The current research trend uses acid aqueous solutions or alkaline aqueous solutions for degradation, which will lead to the problems such as subsequent treatment of the large amount of wastewater, and it is not friendly to the industrial applications and the environment. However, in terms of the current technology, the usage of the organic substance for degradation is limited by the limited content of the structural ester groups, and a relatively high degradation temperature must be applied, which has the disadvantages of energy consumption and poor efficiency, so there are still considerable application defects.

In view of this, how to synthesize the cured product with the chemical degradability and can reuse the waste is the goal of the relevant industry.

### SUMMARY

One object of the present disclosure is to provide a carbonate-containing unsaturated compound and a preparation method therefor, which is prepared by using a carbonate compound as a raw material, and reacting with an epoxy compound or an alcohol compound containing an unsaturated double bond, respectively.

Another object of the present disclosure is to provide a cured product and a method for degrading a cured product, wherein the carbonate-containing unsaturated compound is performed a curing reaction to prepare the cured product, and the cured product can be degraded, so that the product can be recycled and reused to reduce the environmental burden.

According to one embodiment of the present disclosure is to provide a carbonate-containing unsaturated compound, which includes a structure represented by formula (I) or formula (II):

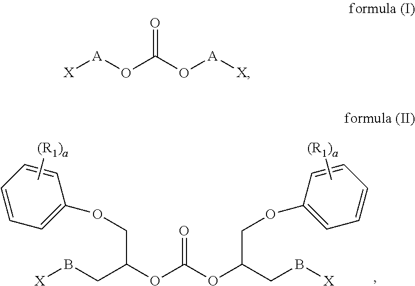

wherein X is a vinyl group, an allyl group, an acrylate or a methacrylate. R₁ is an alkyl group of 1 to 4 carbon atoms, a methoxy group, a nitro group or a halogen atom, and a is an integer from 0 to 5. A is an alkyl group of 1 to 12 carbon atoms, an alkoxy group of 1 to 12 carbon atoms or a structure represented by formula (i):

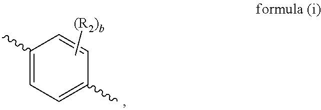

wherein R₂ is the alkyl group of 1 to 4 carbon atoms, the methoxy group, the nitro group or the halogen atom, and b is an integer from 0 to 4. B is the alkyl group of 1 to 12 carbon atoms, the alkoxy group of 1 to 12 carbon atoms, the structure represented by formula (i), a structure represented by formula (ii) or formula (iii):

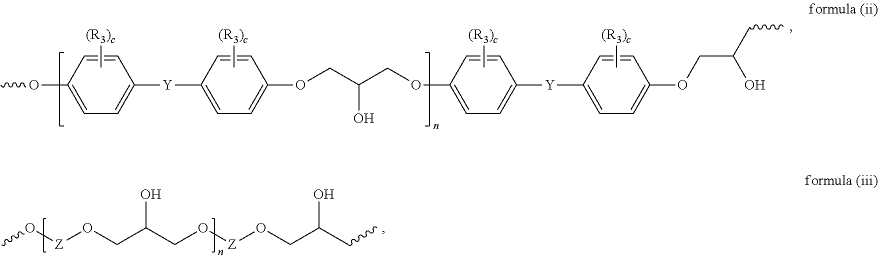

wherein R₃ is the alkyl group of 1 to 4 carbon atoms, the methoxy group, the nitro group or the halogen atom, and c is an integer from 0 to 4. Y is a single bond, the alkyl group of 1 to 12 carbon atoms, a cycloalkyl group of 1 to 12 carbon atoms, an oxygen atom, a sulfur atom, a sulfonyl group, a thionyl group, an acyl group, a fluorenyl group or a hexafluoropropane group. Z is the alkyl group of 1 to 12 carbon atoms, the alkoxy group of 1 to 12 carbon atoms, an ortho-phenylene group, a meta-phenylene group or a para-phenylene group. n is an integer from 0 to 10.

According to another embodiment of the present disclosure is to provide a preparation method for a carbonate-containing unsaturated compound, which includes providing a carbonate-containing compound, providing an unsaturated double bond-containing compound and performing a catalyzing step. The carbonate-containing compound is a dimethyl carbonate or a diphenyl carbonate. The unsaturated double bond-containing compound is an unsaturated double bond-containing monofunctional alcohol compound or an unsaturated double bond-containing monofunctional epoxy compound. The catalyzing step is that the carbonate-containing compound reacted with the unsaturated double bond-containing compound by using a catalyst to obtain the carbonate-containing unsaturated compound, which includes a structure represented by formula (I) or formula (II):

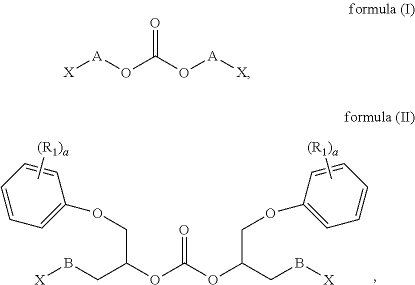

wherein X is a vinyl group, an allyl group, an acrylate or a methacrylate. R₁ is an alkyl group of 1 to 4 carbon atoms, a methoxy group, a nitro group or a halogen atom, and a is an integer from 0 to 5. A is an alkyl group of 1 to 12 carbon atoms, an alkoxy group of 1 to 12 carbon atoms or a structure represented by formula (i):

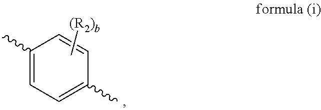

wherein R₂ is the alkyl group of 1 to 4 carbon atoms, the methoxy group, the nitro group or the halogen atom, and b is an integer from 0 to 4. B is the alkyl group of 1 to 12 carbon atoms, the alkoxy group of 1 to 12 carbon atoms, the structure represented by formula (i), a structure represented by formula (ii) or formula (iii):

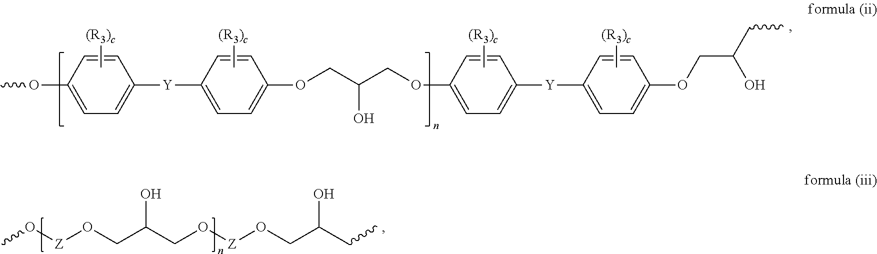

wherein R₃ is the alkyl group of 1 to 4 carbon atoms, the methoxy group, the nitro group or the halogen atom, and c is an integer from 0 to 4. Y is a single bond, the alkyl group of 1 to 12 carbon atoms, a cycloalkyl group of 1 to 12 carbon atoms, an oxygen atom, a sulfur atom, a sulfonyl group, a thionyl group, an acyl group, a fluorenyl group or a hexafluoropropane group. Z is the alkyl group of 1 to 12 carbon atoms, the alkoxy group of 1 to 12 carbon atoms, an ortho-phenylene group, a meta-phenylene group or a para-phenylene group. n is an integer from 0 to 10.

According to the preparation method for the carbonate-containing unsaturated compound of the foregoing aspect, wherein the catalyst can be an ionic liquid or an organic base.

According to the preparation method for the carbonate-containing unsaturated compound of the foregoing aspect, wherein an equivalence ratio of the carbonate-containing compound to the unsaturated double bond-containing compound can be 0.8 to 1.2.

According to further embodiment of the present disclosure is to provide a preparation method for a carbonate-containing unsaturated compound, which includes performing a catalyzing step and performing an adding step. The catalyzing step is that a diphenyl carbonate reacted with a bifunctional epoxy compound by using a catalyst to obtain a reactant. The adding step is that an acrylic acid or a methacrylic acid added in the reactant to obtain the carbonate-containing unsaturated compound, which includes a structure represented by formula (II):

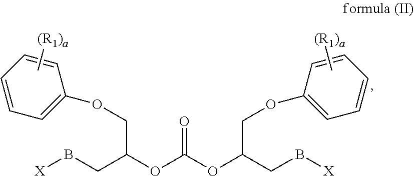

wherein X is a vinyl group, an allyl group, an acrylate or a methacrylate. R₁ is an alkyl group of 1 to 4 carbon atoms, a methoxy group, a nitro group or a halogen atom, and a is an integer from 0 to 5. B is an alkyl group of 1 to 12 carbon atoms, an alkoxy group of 1 to 12 carbon atoms, a structure represented by formula (i), formula (ii) or formula (iii):

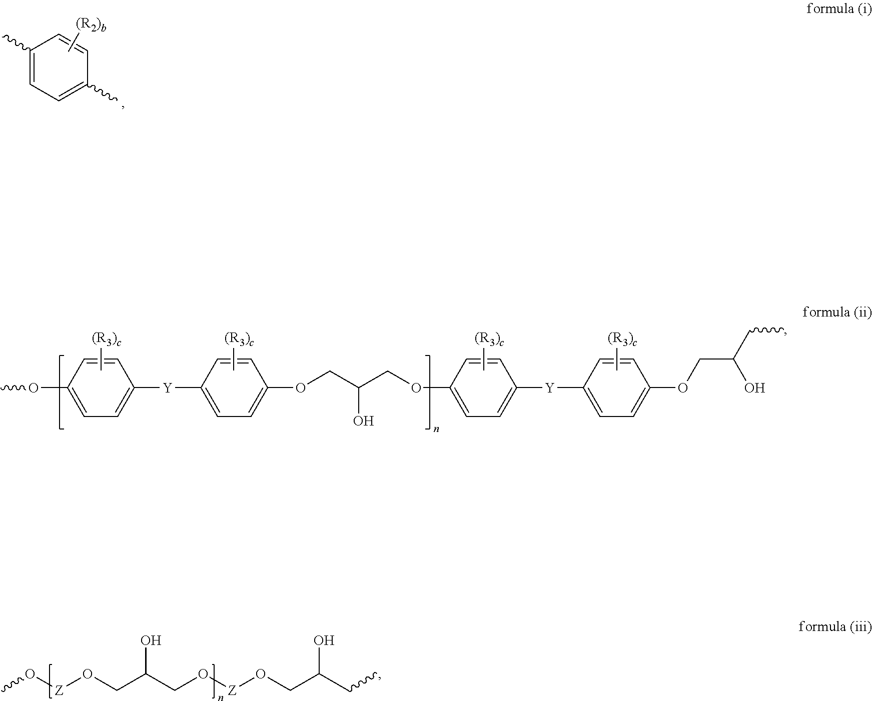

wherein R₂ is the alkyl group of 1 to 4 carbon atoms, the methoxy group, the nitro group or the halogen atom, and b is an integer from 0 to 4. R₃ is the alkyl group of 1 to 4 carbon atoms, the methoxy group, the nitro group or the halogen atom, and c is an integer from 0 to 4. Y is a single bond, the alkyl group of 1 to 12 carbon atoms, a cycloalkyl group of 1 to 12 carbon atoms, an oxygen atom, a sulfur atom, a sulfonyl group, a thionyl group, an acyl group, a fluorenyl group or a hexafluoropropane group. Z is the alkyl group of 1 to 12 carbon atoms, the alkoxy group of 1 to 12 carbon atoms, an ortho-phenylene group, a meta-phenylene group or a para-phenylene group. n is an integer from 0 to 10.

According to the preparation method for the carbonate-containing unsaturated compound of the foregoing aspect, wherein the catalyst can be an organic base, and an equivalence ratio of the bifunctional epoxy compound to the diphenyl carbonate can be 2.0 to 8.0.

According to still another aspect of the present disclosure is to provide a cured product, which is obtained by performing a curing reaction with the carbonate-containing unsaturated compound according to the aforementioned aspect.

According to the cured product of the foregoing aspect, wherein the curing reaction is completed by heating after the carbonate-containing unsaturated compound added to a resin.

According to the cured product of the foregoing aspect, wherein the resin can be an unsaturated polyester resin or a vinyl ester resin.

According to the cured product of the foregoing aspect, wherein an added amount of the carbonate-containing unsaturated compound can be ranged from 3% by weight to 20% by weight of an amount of the resin.

According to yet another aspect of the present disclosure is to provide a method for degrading a cured product, which includes providing the cured product according to the aforementioned aspect and performing a degrading step, wherein the degrading step is that the cured product degraded by reacting an amine compound with the cured product.

Therefore, the carbonate-containing unsaturated compound of the present disclosure can participate in the free radical copolymerization of the unsaturated resin or the vinyl ester resin. By introducing the highly active carbonate structure into the network system, the ester group density of the main structure is increased, giving the material good degradability. Furthermore, a degradation method with the mild conditions is proposed, which can achieve the high degradation efficiency and produce no waste water to facilitate the industrialization and avoid the derived environmental hazard issues.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a preparation method for a carbonate-containing unsaturated compound according to one embodiment of the present disclosure;

FIG. 2 is a flow chart of a preparation method for a carbonate-containing unsaturated compound according to another embodiment of the present disclosure;

FIG. 3 is a flow chart of a preparation method for a cured product according to further another embodiment of the present disclosure; and

FIG. 4 is a flow chart of a method for degrading a cured product according to yet another embodiment of the present disclosure.

### DETAILED DESCRIPTION

The present disclosure will be further exemplified by the following specific embodiments. However, the embodiments can be applied to various inventive concepts and can be embodied in various specific ranges. The specific embodiments are only for the purposes of description, and are not limited to these practical details thereof.

In the present disclosure, the compound structure can be represented by a skeleton formula, and the representation can omit the carbon atom, the hydrogen atom and the carbon-hydrogen bond. In the case that the functional group is depicted clearly in the structural formula, the depicted one is preferred.

In the present disclosure, in order to concise and smooth, “carbonate-containing unsaturated compound, comprising a structure represented by formula (I)” can be represented as a carbonate-containing unsaturated compound represented by formula (I) or a carbonate-containing unsaturated compound (I) in some cases, and the other compounds or groups can be represented in the same manner.

### <Carbonate-Containing Unsaturated Compound>

A carbonate-containing unsaturated compound is provided of the present disclosure, which includes a structure represented by formula (I) or formula (II):

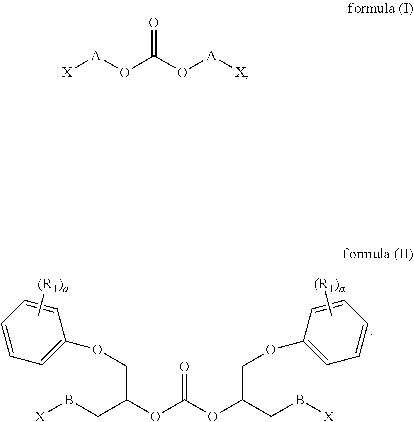

wherein X is a vinyl group, an allyl group, an acrylate or a methacrylate. R₁ is an alkyl group of 1 to 4 carbon atoms, a methoxy group, a nitro group or a halogen atom, and a is an integer from 0 to 5. A is an alkyl group of 1 to 12 carbon atoms, an alkoxy group of 1 to 12 carbon atoms or a structure represented by formula (i):

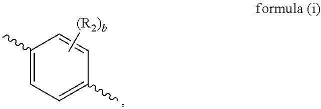

wherein R₂ is the alkyl group of 1 to 4 carbon atoms, the methoxy group, the nitro group or the halogen atom, and b is an integer from 0 to 4. B is the alkyl group of 1 to 12 carbon atoms, the alkoxy group of 1 to 12 carbon atoms, the structure represented by formula (i), a structure represented by formula (ii) or formula (iii):

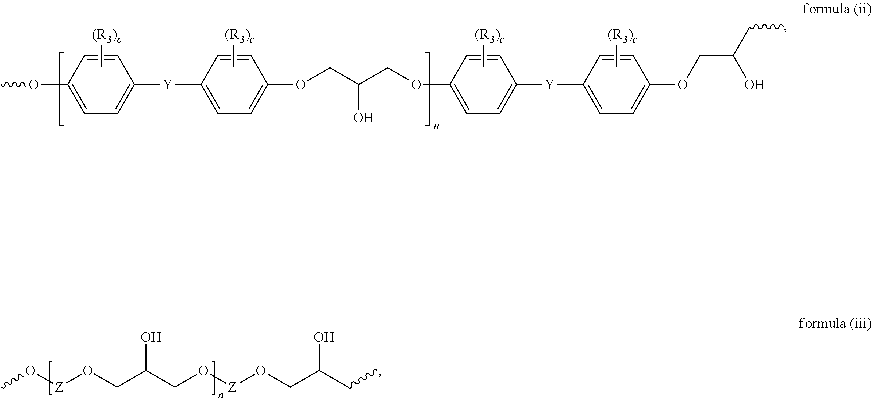

wherein R₃ is the alkyl group of 1 to 4 carbon atoms, the methoxy group, the nitro group or the halogen atom, and c is an integer from 0 to 4. Y is a single bond, the alkyl group of 1 to 12 carbon atoms, a cycloalkyl group of 1 to 12 carbon atoms, an oxygen atom, a sulfur atom, a sulfonyl group, a thionyl group, a carbonyl group, a fluorene group or a hexafluoropropane group. Z is the alkyl group of 1 to 12 carbon atoms, the alkoxy group of 1 to 12 carbon atoms, an ortho-phenylene group, a meta-phenylene group or a para-phenylene group. n is an integer from 0 to 10.

Therefore, the carbonate-containing unsaturated compound of the present disclosure is given the thermosetting product degradability by introducing the carbonate ester structure, and the purpose of recycling and degrading the material can be achieved in the future.

### <Preparation Method for Carbonate-Containing Unsaturated Compound>

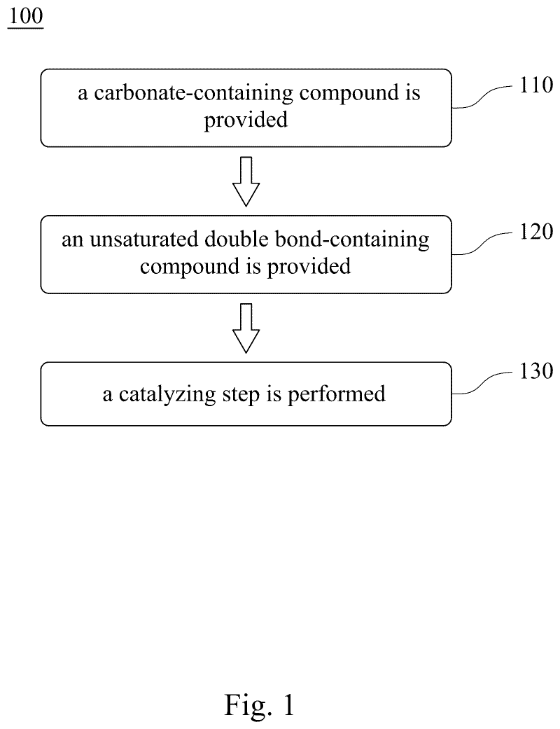

Reference is made to FIG. 1, which is a flow chart of a preparation method for a carbonate-containing unsaturated compound 100 according to one embodiment of the present disclosure. In FIG. 1, the preparation method for the carbonate-containing unsaturated compound 100 includes a step 110, a step 120 and a step 130.

In the step 110, a carbonate-containing compound is provided, wherein the carbonate-containing compound is a dimethylcarbonate (DMC) or a diphenylcarbonate (DPC).

In the step 120, an unsaturated double bond-containing compound is provided, wherein the unsaturated double bond-containing compound is an unsaturated double bond-containing monofunctional alcohol compound or an unsaturated double bond-containing monofunctional epoxy compound.

In the step 130, a catalyzing step is performed, wherein the carbonate-containing compound is reacted with the unsaturated double bond-containing compound by using a catalyst to obtain the carbonate-containing unsaturated compound, which includes a structure represented by formula (I) or formula (II):

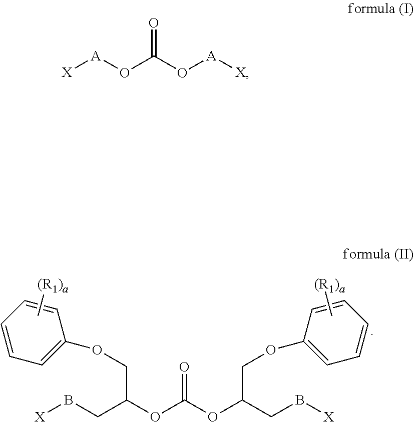

The definition of X, A, B, R₁ and a can refer to the aforementioned paragraph, and will not be described herein. The aforementioned catalyst can be an ionic liquid or an organic base.

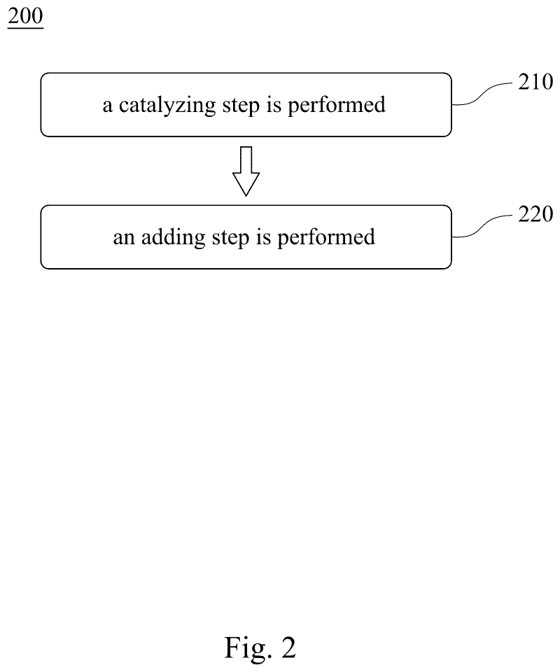

Reference is made to FIG. 2, which is a flow chart of a preparation method for a carbonate-containing unsaturated compound 200 according to another embodiment of the present disclosure. In FIG. 2, the preparation method for the carbonate-containing unsaturated compound 200 includes a step 210 and a step 220.

In the step 210, a catalyzing step is performed, wherein a diphenyl carbonate is reacted with a bifunctional epoxy compound by using a catalyst to obtain a reactant. The aforementioned catalyst can be an organic base.

In the step 220, an adding step is performed, wherein an acrylic acid or a methacrylic acid is added in the reactant to obtain the carbonate-containing unsaturated compound, which includes a structure represented by formula (II):

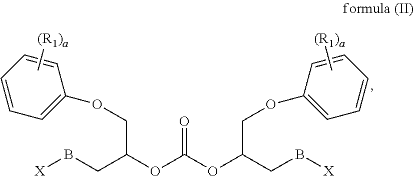

the definition of X, B, R₁ and a can refer to the aforementioned paragraph, and will not be described herein.

Specifically, when the carbonate-containing unsaturated compound is the structure represented by formula (I), the preparation method thereof is to react the dimethyl carbonate with the unsaturated double bond-containing monofunctional alcohol compound at an equivalent ratio of 0.8 to 1.2, and the ionic liquid is used as the catalyst. An added amount of the catalyst can be ranged from 0.01% by weight to 2% by weight of a total content of the reactant, and a reaction temperature is 60° C. to 90° C.

Furthermore, when the carbonate-containing unsaturated compound is the structure represented by formula (II), which has the two preparation methods. The first preparation method is to react the diphenyl carbonate with the unsaturated double bond-containing monofunctional epoxy compound at an equivalent ratio of 0.8 to 1.2, and the organic base is used as the catalyst. An added amount of the catalyst can be ranged from 0.01% by weight to 2% by weight of a content of the unsaturated double bond-containing monofunctional epoxy compound, and a reaction temperature is 80° C. to 140° C. The second preparation method is to react the bifunctional epoxy compound with the diphenyl carbonate at an equivalent ratio of 2.0 to 8.0, and the organic base is used as the catalyst. An added amount of the catalyst can be ranged from 0.01% by weight to 2% by weight of a content of the bifunctional epoxy compound, and then the acrylic acid or the methacrylic acid is added for the reaction, wherein an equivalence ratio of the acrylic acid or the methacrylic acid to the bifunctional epoxy compound is 0.4 to 0.6, and a reaction temperature is 80° C. to 140° C.

### <Cured Product>

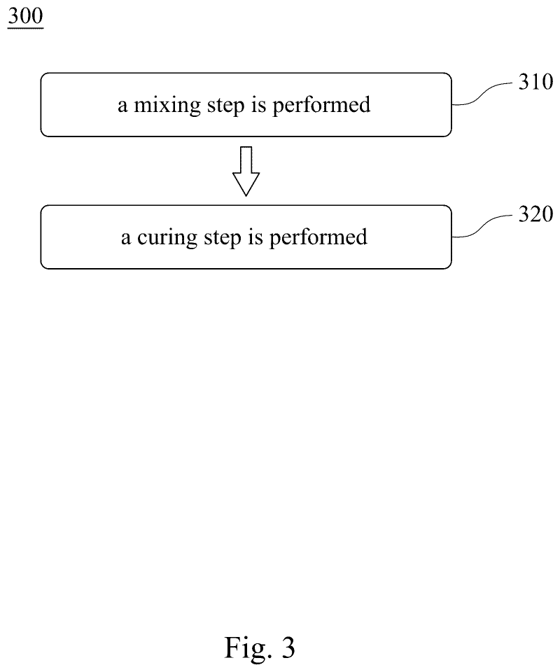

A cured product is further provided of the present disclosure, which is obtained by performing a curing reaction with the carbonate-containing unsaturated compound according to the aforementioned aspect. The aforementioned curing reaction is referred with FIG. 3, wherein FIG. 3 is a flow chart of a preparation method for a cured product 300 according to further another embodiment of the present disclosure. In FIG. 3, the preparation method for the cured product 300 includes a step 310 and a step 320.

In the step 310, a mixing step is performed, wherein the carbonate-containing unsaturated compound is added to a resin. Specifically, an added amount of the carbonate-containing unsaturated compound is ranged from 3% by weight to 20% by weight of an amount of the resin, which can improve the degradability of the cured product without affecting the basic physical properties. The detail of the carbonate-containing unsaturated compound can refer to the aforementioned paragraph, and will not be described herein. The aforementioned resin can be but not limited to an unsaturated polyester resin or a vinyl ester resin.

In the step 320, a curing step is performed, wherein the carbonate-containing unsaturated compound and the resin are performed the free radical copolymerization to form a cured product, and a curing temperature of heating can be 25° C. to 80° C. The curing temperature of heating and the heating time can be appropriately adjusted according to the type of the used carbonate-containing unsaturated compound and the resin, and the present disclosure is not limited thereto.

### <Method for Degrading Cured Product>

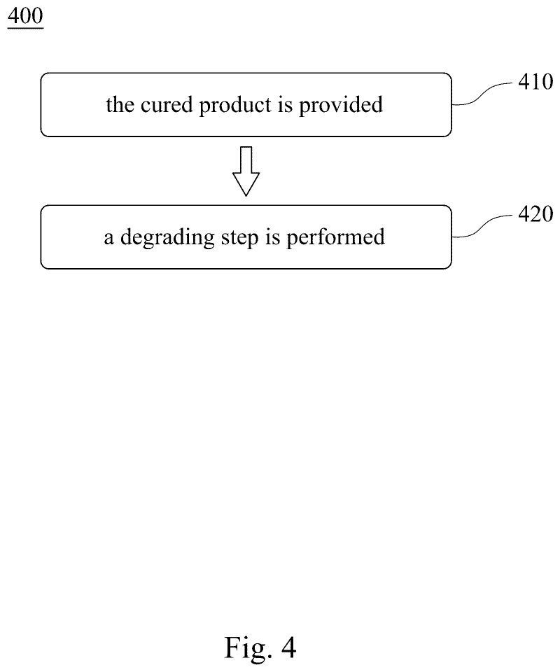

Reference is made to FIG. 4, which is a flow chart of a method for degrading a cured product 400 according to yet another embodiment of the present disclosure. In FIG. 4, the method for degrading the cured product 400 includes a step 410 and a step 420.

In the step 410, the aforementioned cured product is provided. In the step 420, a degrading step is performed, wherein the cured product is degraded by reacting an amine compound with the aforementioned cured product. Specifically, the aforementioned degrading step can be degraded at 80° C. to 150° C. without adding any catalyst, and the liquid after the degrading step can be purified by distillation, therefore the amine compound can be reused. Furthermore, the produced urea derivative can be recovered, and can be further used in the coatings or the polyurethane materials to achieve the goal of recycling applications.

The present disclosure will be further exemplified by the following specific embodiments so as to facilitate utilizing and practicing the present disclosure completely by the people skilled in the art without over-interpreting and over-experimenting. However, the readers should understand that the present disclosure should not be limited to these practical details thereof, that is, these practical details are used to describe how to implement the materials and methods of the present disclosure and are not necessary.

### Example/Comparative Example

### <Preparation of Carbonate-Containing Unsaturated Compound>

Example 1:10 g of dimethyl carbonate and 28.89 g of 2-hydroxyethyl methacrylate (HEMA) were mixed at the equivalence ratio of 1:1 to form the reactant. Next, adding trioctylmethylphosphonium methyl carbonate (P₈₈₈₁CH₃OCOO) ionic liquid of 0.5 weight percent of the total amount of the reactant, and heating to 80° C. for 8 hours to obtain the carbonate-containing unsaturated compound of Example 1, and the yield thereof was about 60%. Data from FTIR spectra: 1748 cm⁻¹ (carbonate C═O), 1715 cm⁻¹ (acrylate C═O). The reaction equation of Example 1 is shown in Table 1.

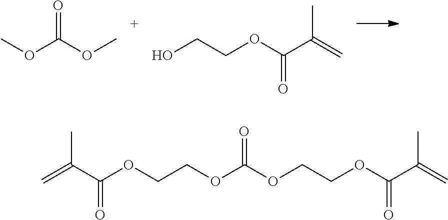

Example 2:10 g of diphenyl carbonate and 13.27 g of glycidyl methacrylate (GMA) were mixed at the equivalence ratio of 1:1, and in the nitrogen environment at 110° C. to form the homogeneous solution. Next, adding 0.027 g of pyridine (0.2 wt % of GMA) and reacting for 3 hours to obtain the carbonate-containing unsaturated compound of Example 2, and the yield thereof was about 90%. Data from FTIR spectra: 1749 cm⁻¹ (aliphatic carbonate C═O), 1715 cm⁻¹ (acrylate C═O). The reaction equation of Example 2 is shown in Table 2.

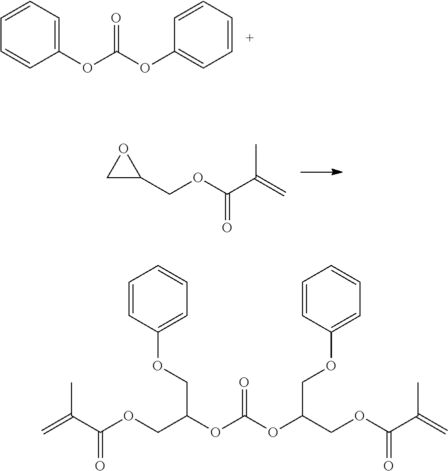

Example 3:10 g of diphenyl carbonate and 34.54 g of bisphenol A diglycidyl ether (commodity code BE188 from CHANG CHUN PLASTICS CO., LTD.) were mixed at the equivalence ratio of 1:2, and in the nitrogen environment at 110° C. to form the homogeneous solution. Next, adding 0.1727 g of pyridine (0.5 wt % of BE188) and reacting for 3 hours. Then, adding 8.04 g of methacrylic acid, wherein the equivalence ratio of the methacrylic acid to BE188 was 0.5:1, and reacting for 4 hours to obtain the carbonate-containing unsaturated compound of Example 3. Data from FTIR spectra: 1749 cm⁻¹ (carbonate C═O), 1719 cm⁻¹ (acrylate C═O). The reaction equation of Example 3 is shown in Table 3.

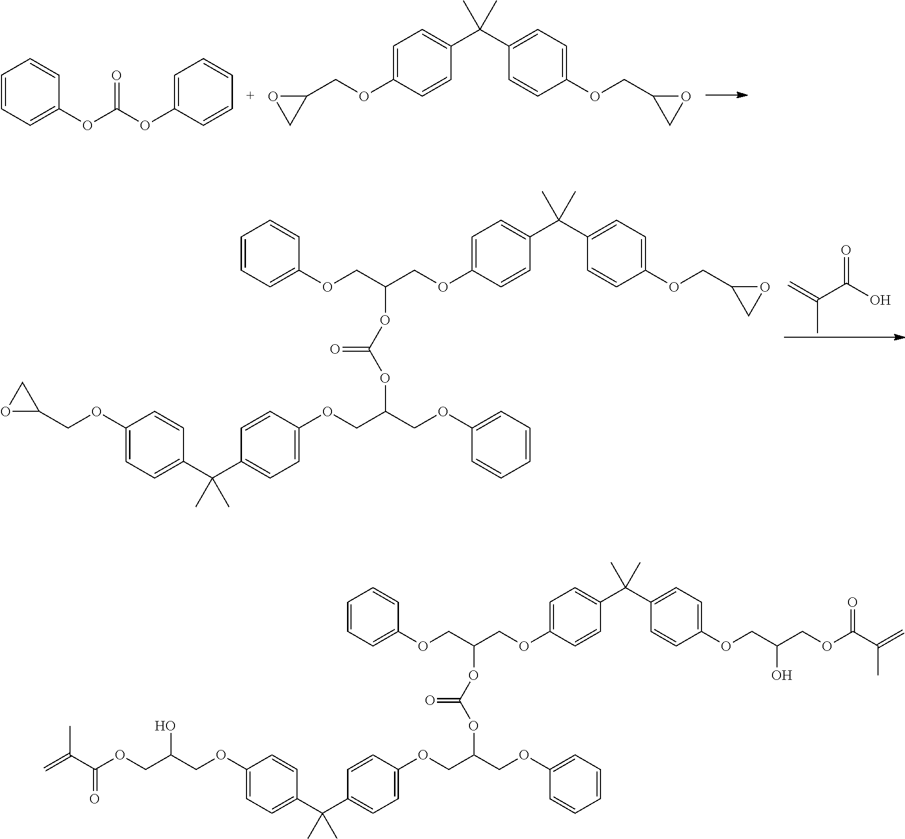

### <Preparation of Cured Product>

The carbonate-containing unsaturated compound of Example 1 to Example 3 were added to the unsaturated polyester resin (UP1) or bisphenol A vinyl ester resin (VE1), respectively, and then diluted with the styrene (SM). Next, adding 1 phr of peroxide MEKPO and 1 phr of cobalt octoate, and stirring evenly and pouring into the mold to cure at the room temperature for 12 hours. Then, the cured product of Example 4 to Example 9 and Comparative Example 1 to Comparative Example 4 were obtained by curing at 80° C. for 4 hours.

The formulation and the content used for Example 4 to Example 9 and Comparative Example 1 to Comparative Example 4 are shown in Table 4.

TABLE 4

| | Example 1 | Example 2 | Example 3 | UP1 | VE1 | SM |
|---|---|---|---|---|---|---|
| Example 4 | 15 | 0 | 0 | 100 | 0 | 5 |
| Example 5 | 0 | 10 | 0 | 100 | 0 | 8 |
| Example 6 | 0 | 0 | 8 | 100 | 0 | 14 |
| Example 7 | 15 | 0 | 0 | 0 | 100 | 5 |
| Example 8 | 0 | 10 | 0 | 0 | 100 | 8 |
| Example 9 | 0 | 0 | 8 | 0 | 100 | 14 |
| Comparative | 0 | 0 | 0 | 100 | 0 | 10 |
| Example 1 | | | | | | |
| Comparative | 0 | 0 | 0 | 0 | 100 | 10 |
| Example 2 | | | | | | |
| Comparative | 0 | 0 | 2 | 100 | 0 | 10 |
| Example 3 | | | | | | |
| Comparative | 0 | 0 | 22 | 100 | 0 | 25 |
| Example 4 | | | | | | |

### <Thermal Property Evaluation>

Example 4 to Example 9 and Comparative Example 1 to Comparative Example 4 were performed the thermal property evaluation, which used the differential scanning calorimeter (DSC) to measure the glass transition temperature (T_{g}) at a heating rate of 10° C./min, and the measurement results of the T_{g} (C) are shown in Table 5.

TABLE 5

| | Example 4 | Example 5 | Example 6 | Example 7 | Example 8 |
|---|---|---|---|---|---|
| T_{g} | 115 | 114 | 117 | 123 | 120 |
| | | Comparative | Comparative | Comparative | Comparative |
| | Example 9 | Example 1 | Example 2 | Example 3 | Example 4 |
| T_{g} | 125 | 109 | 120 | 111 | 73 |

As shown in Table 5, the glass transition temperature of Comparative Example 4 is lower because the molecular weight of the carbonate-containing unsaturated compound of Example 3 is larger and the viscosity is higher, so that after adding Example 3, the mixing viscosity will increase significantly. Therefore, if the addition ratio of Example 3 is too high, it will lead to the need to add more diluent SM to achieve the required operating viscosity that is unfavorable for the physical properties of the cured product. However, the suitable operating viscosities of Example 4 to Example 9 are achieved by the adjustment of the addition ratio of the diluent SM and the carbonate-containing unsaturated compound, and the good glass transition temperature is maintained.

### <Degradation of Cured Product>

The cured product of the present disclosure can be performed the degradation reaction by the amine compound. First, 0.2 g of the cured product of Example 4 to Example 9 and Comparative Example 1 to Comparative Example 4 were placed in the container respectively with 4 g of hexylamine, and heated to 130° C. in the oven. After heating for 24 hours, the remaining solid was taken out to measure the residual amount, and the residual weight (%) is listed in Table 6.

TABLE 6

| | Example 4 | Example 5 | Example 6 | Example 7 | Example 8 |
|---|---|---|---|---|---|
| Residual | 0 | 0 | 0 | 0 | 0 |
| weight | | | | | |
| | | Comparative | Comparative | Comparative | Comparative |
| | Example 9 | Example 1 | Example 2 | Example 3 | Example 4 |
| Residual | 0 | 78 | 95 | 54 | 0 |
| weight | | | | | |

As shown in Table 6, Example 4 to Example 9 can effectively provide a degradation site by introducing the carbonate group into the network structure, so that the disintegration efficiency of the network structure can be significantly increased, and the effect of complete degradation can be achieved at 130° C. without the catalyst. Furthermore, Comparative Example 1 and Comparative Example 2 do not add the carbonate-containing unsaturated compound of the present disclosure, so that the degradation effect thereof is not good. However, in Comparative Example 3, due to the content of the carbonate-containing unsaturated compound is low, the purpose of complete degradation is not achieved, but it is still observed that the addition of the carbonate-containing unsaturated compound of the present disclosure has a significant improvement in degradability.

In conclusion, the carbonate-containing unsaturated compound of the present disclosure is obtained by using the carbonate compound and the epoxy compound or the alcohol compound that contains unsaturated double bond, which can be introduced into the commercially unsaturated polyester resin or vinyl ester resin for enhancing the degradability of the cured product. Furthermore, the cured product can be degraded by using the amine compound under the mild condition to response to the issue of the thermoset recycling.

Although the present disclosure has been described in the above by way of embodiments, it is not intended to limit the present disclosure. Persons having ordinary skill in the art can make various changes and modifications without departing from the spirit and scope of the present disclosure. Therefore, the scope of protection of the present disclosure should be defined by the scope of the following claims.

### DENOTATION OF REFERENCE NUMERALS

100,200: preparation method for a carbonate-containing unsaturated compound
300: preparation method for a cured product
400: method for degrading a cured product
110,120,130,210,220,310,320,410,420: step

1. A carbonate-containing unsaturated compound, comprising a structure represented by formula (I) or formula (II): 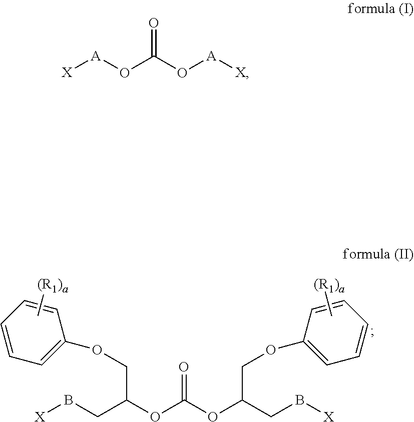
wherein X is a vinyl group, an allyl group, an acrylate or a methacrylate;
wherein R₁ is an alkyl group of 1 to 4 carbon atoms, a methoxy group, a nitro group or a halogen atom, and a is an integer from 0 to 5;
wherein A is an alkyl group of 1 to 12 carbon atoms, an alkoxy group of 1 to 12 carbon atoms or a structure represented by formula (i): 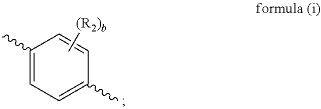
wherein R₂ is the alkyl group of 1 to 4 carbon atoms, the methoxy group, the nitro group or the halogen atom, and b is an integer from 0 to 4;
wherein B is the alkyl group of 1 to 12 carbon atoms, the alkoxy group of 1 to 12 carbon atoms, the structure represented by formula (i), a structure represented by formula (ii) or formula (iii): 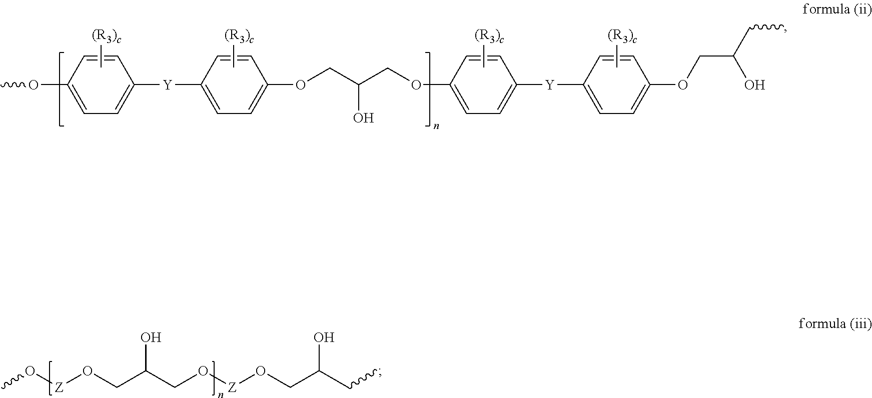
wherein R₃ is the alkyl group of 1 to 4 carbon atoms, the methoxy group, the nitro group or the halogen atom, and c is an integer from 0 to 4;
wherein Y is a single bond, the alkyl group of 1 to 12 carbon atoms, a cycloalkyl group of 3 to 12 carbon atoms, an oxygen atom, a sulfur atom, a sulfonyl group, a thionyl group, an acyl group, a fluorenyl group or a hexafluoropropane group;
wherein Z is the alkyl group of 1 to 12 carbon atoms, the alkoxy group of 1 to 12 carbon atoms, an ortho-phenylene group, a meta-phenylene group or a para-phenylene group; and
wherein n is an integer from 0 to 10.

2. A preparation method for a carbonate-containing unsaturated compound, comprising:
providing a carbonate-containing compound, which is a dimethyl carbonate or a diphenyl carbonate;
providing an unsaturated double bond-containing compound, which is an unsaturated double bond-containing monofunctional alcohol compound or an unsaturated double bond-containing monofunctional epoxy compound; and
performing a catalyzing step, wherein the carbonate-containing compound is reacted with the unsaturated double bond-containing compound by using a catalyst to obtain the carbonate-containing unsaturated compound, which comprises a structure represented by formula (I) or formula (II): 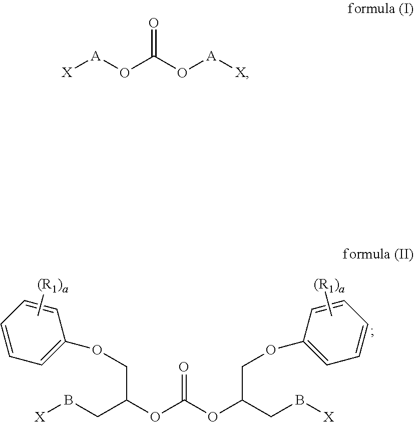
wherein X is a vinyl group, an allyl group, an acrylate or a methacrylate;
wherein R₁ is an alkyl group of 1 to 4 carbon atoms, a methoxy group, a nitro group or a halogen atom, and a is an integer from 0 to 5;
wherein A is an alkyl group of 1 to 12 carbon atoms, an alkoxy group of 1 to 12 carbon atoms or a structure represented by formula (i): 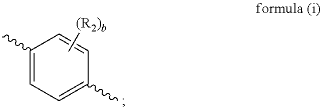
wherein R₂ is the alkyl group of 1 to 4 carbon atoms, the methoxy group, the nitro group or the halogen atom, and b is an integer from 0 to 4;
wherein B is the alkyl group of 1 to 12 carbon atoms, the alkoxy group of 1 to 12 carbon atoms, the structure represented by formula (i), a structure represented by formula (ii) or formula (iii): 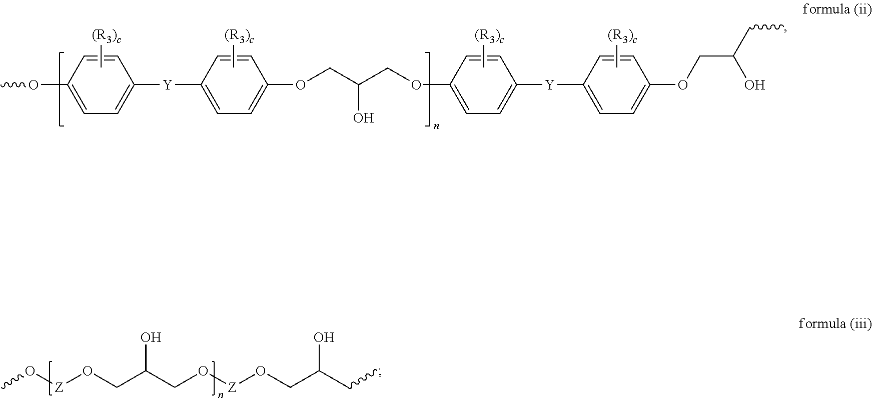
wherein R₃ is the alkyl group of 1 to 4 carbon atoms, the methoxy group, the nitro group or the halogen atom, and c is an integer from 0 to 4;
wherein Y is a single bond, the alkyl group of 1 to 12 carbon atoms, a cycloalkyl group of 3 to 12 carbon atoms, an oxygen atom, a sulfur atom, a sulfonyl group, a thionyl group, an acyl group, a fluorenyl group or a hexafluoropropane group;
wherein Z is the alkyl group of 1 to 12 carbon atoms, the alkoxy group of 1 to 12 carbon atoms, an ortho-phenylene group, a meta-phenylene group or a para-phenylene group;
wherein n is an integer from 0 to 10.

3. The preparation method for the carbonate-containing unsaturated compound of claim 2, wherein the catalyst is an ionic liquid or an organic base.

4. The preparation method for the carbonate-containing unsaturated compound of claim 2, wherein an equivalence ratio of the carbonate-containing compound to the unsaturated double bond-containing compound is 0.8 to 1.2.

5. A preparation method for a carbonate-containing unsaturated compound, comprising:
performing a catalyzing step, wherein a diphenyl carbonate is reacted with a bifunctional epoxy compound by using a catalyst to obtain a reactant; and
performing an adding step, wherein an acrylic acid or a methacrylic acid is added in the reactant to obtain the carbonate-containing unsaturated compound, which comprises a structure represented by formula (II): 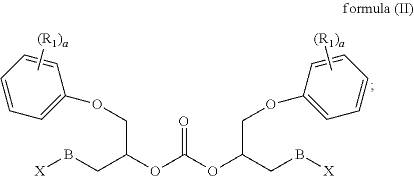
wherein X is a vinyl group, an allyl group, an acrylate or a methacrylate;
wherein R₁ is an alkyl group of 1 to 4 carbon atoms, a methoxy group, a nitro group or a halogen atom, and a is an integer from 0 to 5;
wherein B is an alkyl group of 1 to 12 carbon atoms, an alkoxy group of 1 to 12 carbon atoms, a structure represented by formula (i), formula (ii) or formula (iii): 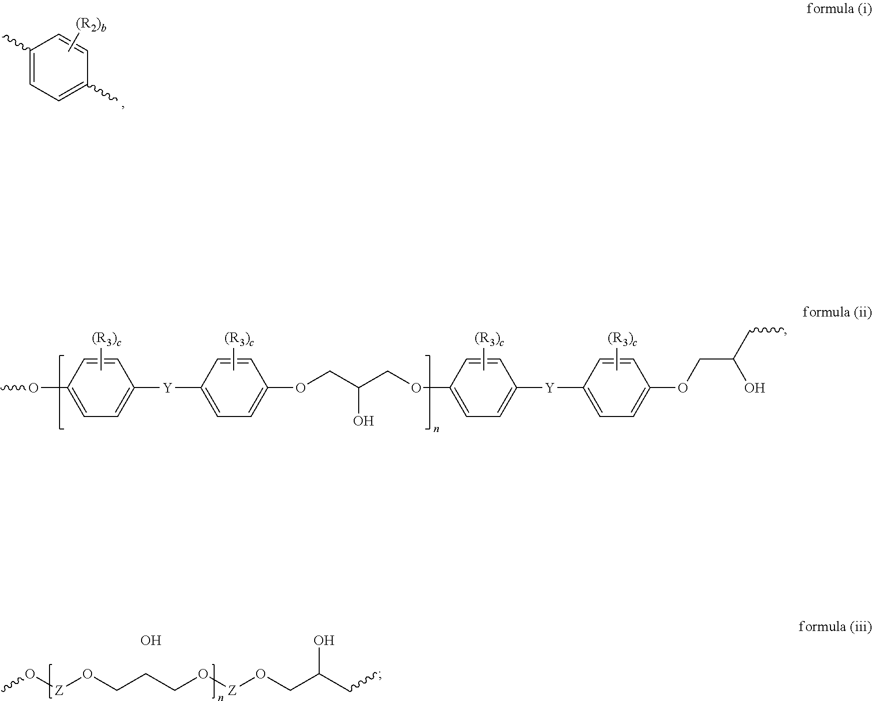
wherein R₂ is the alkyl group of 1 to 4 carbon atoms, the methoxy group, the nitro group or the halogen atom, and b is an integer from 0 to 4;
wherein R₃ is the alkyl group of 1 to 4 carbon atoms, the methoxy group, the nitro group or the halogen atom, and c is an integer from 0 to 4;
wherein Y is a single bond, the alkyl group of 1 to 12 carbon atoms, a cycloalkyl group of 3 to 12 carbon atoms, an oxygen atom, a sulfur atom, a sulfonyl group, a thionyl group, an acyl group, a fluorenyl group or a hexafluoropropane group;
wherein Z is the alkyl group of 1 to 12 carbon atoms, the alkoxy group of 1 to 12 carbon atoms, an ortho-phenylene group, a meta-phenylene group or a para-phenylene group;
wherein n is an integer from 0 to 10.

6. The preparation method for the carbonate-containing unsaturated compound of claim 5, wherein the catalyst is an organic base, and an equivalence ratio of the bifunctional epoxy compound to the diphenyl carbonate is 2.0 to 8.0.

7. A cured product, which is obtained by performing a curing reaction with the carbonate-containing unsaturated compound of claim 1.

8. The cured product of claim 7, wherein the curing reaction is completed by heating after the carbonate-containing unsaturated compound added to a resin.

9. The cured product of claim 8, wherein the resin is an unsaturated polyester resin or a vinyl ester resin.

10. The cured product of claim 8, wherein an added amount of the carbonate-containing unsaturated compound is ranged from 3% by weight to 20% by weight of an amount of the resin.

11. A method for degrading a cured product, comprising:
providing the cured product of claim 7; and
performing a degrading step, wherein the cured product is degraded by reacting an amine compound with the cured product.
